(19) United States
(12) Patent Application Publication
(10) Pub. No.: US 2012/0095238 A1
(43) Pub. Date: Apr. 19, 2012

(54) Continuous Method For Producing Amides Of Aromatic Carboxylic Acids

(75) Inventors: Matthias Krull, Harxheim (DE); Roman Morschhaeuser, Mainz (DE)

(73) Assignee: CLARIANT FINANCE (BVI) LIMITED, Tortola (VG)

(21) Appl. No.: 13/378,242

(22) PCT Filed: Jun. 9, 2010
(86) PCT No.: PCT/EP2010/003442

§ 371 Date: Dec. 14, 2011

(30) Foreign Application Priority Data
Jun. 30, 2009 (DE) 10 2009 031 058.4

(51) Int. Cl. C07C 231/02; C07D 209/48; C07D 213/56

(52) U.S. Cl. 548/480; 564/134; 546/316

(57) ABSTRACT

The invention relates to a continuous method for producing amides of aromatic carboxylic acids by reacting at least one carbonic acid ester of formula (I) R³—OOOR⁴, wherein R³ represents an optionally substituted aromatic hydrocarbon group with 5 to 100 carbon atoms and R⁴ represents a hydrocarbon group with 1 to 30 carbon atoms, with at least one amine of formula (II) HNR¹R², wherein R¹ and R² independently represent hydrogen or a hydrocarbon group with 1 to 100 C atoms, in a reaction tube the longitudinal axis of which extends in the direction of propagation of the microwaves of a monomode microwave applicator, under microwave irradiation to form carboxamide.

The present invention relates to a continuous process for preparing amides of aromatic carboxylic acids under microwave irradiation on the industrial scale.

Amides of aromatic carboxylic acids find various uses as chemical raw materials. For instance, various amides are used as intermediates for the production of pharmaceuticals and agrochemicals. In particular, tertiary amides of aromatic carboxylic acids and especially tertiary amides of alkylphenylcarboxylic acids are a class of compounds of great pharmacological and also industrial interest. For example, amides of alkylbenzoic acids with secondary alkylamines find use as insect repellents.

In addition to the esterification of free carboxylic acids with amines, an important way of preparing aromatic carboxamides, especially on the industrial scale, is the reaction of reactive carboxylic acid derivatives, for example of acid chlorides, anhydrides and esters, with the appropriate amines. While the synthesis of amides proceeding from acid chlorides leads to at least equimolar amounts of salts to be disposed of and unwanted residual halide ion contents in the amides, the reactivity especially of the readily obtainable esters of carboxylic acids with aliphatic alcohols toward amines is comparatively low, and so this aminolysis requires long reaction times, high temperatures and/or strongly basic catalysts. Under these reaction conditions, unwanted side reactions, for example oxidation of the amine, thermal disproportionation of secondary amines to primary and tertiary amine and/or decarboxylation of the carboxylic acid, often occur. These impair the properties of the target products, for example the color thereof, lower the yield and often necessitate additional workup steps.

A more recent approach to the synthesis of amines is the microwave-supported reaction of carboxylic esters with amines to give amides. This can also be performed without solvent and thus results not only in increased space-time yields but also in reduced environmental pollution.

Zradni et al. (Synth. Commun. 2002, 32, 3525-3531) disclose the preparation of amides by reaction of esters of aliphatic carboxylic acids with amines in the presence of relatively large, i.e. up to equimolar, amounts of potassium tert-butoxide under the influence of microwaves. This works on the mmol scale.

Perreux et al. (Tetrahedron 59 (2003), 2185-2189) disclose the preparation of carboxamides by microwave-supported aminolysis of carboxylic esters with primary amines. This works with a monomode reactor on the laboratory scale.

Varma et al. (Tetrahedron Letters 40 (1999), 6177-6180) disclose the aminolysis of aromatic carboxylic esters with aryl- and alkylamines in the presence of potassium tert-butoxide in a domestic microwave on the mmol scale. Conversions of secondary amines proceed only slowly.

The scaleup of such microwave-supported aminolyses from the laboratory to an industrial scale and hence the development of plants suitable for production of several tonnes, for example several tens, several hundreds or several thousands of tonnes, per year with space-time yields of interest for industrial scale applications has, however, not been achieved to date. One reason for this is the penetration depth of microwaves into the reaction mixture, which is typically limited to several millimeters to a few centimeters, and causes restriction to small vessels especially in reactions performed in batchwise processes, or leads to very long reaction times in stirred reactors. The occurrence of discharge processes and plasma formation places tight limits on an increase in the field strength, which is desirable for the irradiation of large amounts of substance with microwaves, especially in the multimode units used with preference to date for scaleup of chemical reactions. Moreover, scaleup problems are presented by the inhomogeneity of the microwave field, which leads to local overheating of the reaction mixture in these multimode microwave systems and is caused by more or less uncontrolled reflections of the microwaves injected into the microwave oven at the walls thereof and the reaction mixture. In addition, the microwave absorption coefficient of the reaction mixture, which often changes during the reaction, presents difficulties with regard to a safe and reproducible reaction regime. WO 90/03840 discloses a continuous process for performing various chemical reactions in a continuous laboratory microwave reactor. For example, dimethyl succinate is reacted with ammonia at 135° C. with 51% yield to give succinamide. The yields achieved, and also the reaction volume of 24 ml of the microwave operated in multimode, however, do not allow up-scaling to the industrial scale range. The efficiency of this process with regard to the microwave absorption of the reaction mixture is low due to the more or less homogeneous distribution of microwave energy over the applicator space in multimode microwave applicators, and the lack of focus of the microwave energy on the tube coil. A significant increase in the microwave power injected can lead to unwanted plasma discharges or to what are called thermal runaway effects. In addition, the spatial inhomogeneities of the microwave field in the applicator space, which change with time and are referred to as hotspots, make a reliable and reproducible reaction regime on a large scale impossible.

Additionally known are monomode or single-mode microwave applicators which work with a single wave mode which propagates in only one spatial direction and is focused onto the reaction vessel by waveguides of exact dimensions. This equipment allows higher local field strengths, but has to date been restricted to small reaction volumes (≦50 ml) on the laboratory scale due to the geometric requirements (for example, the intensity of the electrical field is at its greatest at its wave crests and approaches zero at the node points).

A process for preparing amides of aromatic carboxylic acids was therefore sought, in which carboxylic ester and amine can be converted to the amide under microwave irradiation even on the industrial scale. This should achieve very high, i.e. up to quantitative, conversion levels with minimum reaction times. The process should additionally enable a very energy-saving preparation of the carboxamides, which means that the microwave power used should be absorbed very substantially quantitatively by the reaction mixture and the process should give a high energy efficiency. At the same time, only minor amounts, if any, of by-products should be obtained. The amides should also have low intrinsic color. In addition, the process should ensure a reliable and reproducible reaction regime.

It has been found that, surprisingly, amides of aromatic carboxylic acids can be prepared in industrially relevant amounts by reaction of esters of aromatic carboxylic acids with amines in a continuous process by only briefly heating by means of irradiation with microwaves in a reaction tube whose longitudinal axis is in the direction of propagation of the microwaves of a monomode microwave applicator. At the same time, the microwave energy injected into the microwave applicator is absorbed virtually quantitatively by the reaction mixture. The process according to the invention additionally has a high level of reliability in execution and gives high reproducibility of the reaction conditions established. The amides prepared by the process according to the invention exhibit a high purity and low intrinsic color not obtainable in comparison to by conventional preparation processes without additional process steps.

The invention provides a continuous process for preparing amides of aromatic carboxylic acids, in which at least one carboxylic ester of the formula (I)

R³—COOR⁴ (I)

in which

R³ is an optionally substituted aromatic hydrocarbyl radical having 5 to 100 carbon atoms and

R⁴ is an optionally substituted hydrocarbyl radical having 1 to 30 carbon atoms is reacted with at least one amine of the formula (II)

HNR¹R² (II)

in which R¹ and R² are each independently hydrogen or an optionally substituted hydrocarbyl radical having 1 to 100 carbon atoms,

under microwave irradiation in a reaction tube whose longitudinal axis is in the direction of propagation of the microwaves from a monomode microwave applicator to give the carboxamide.

Esters of the formula (I) preferred in accordance with the invention derive from aromatic carboxylic acids of the formula (III)

R³COOH (III)

and alcohols of the formula (IV)

R⁴OH (IV)

where R³ and R⁴ are each as defined above,

from which they can be prepared by known methods, for example by condensation.

Aromatic carboxylic acids (III) are understood here generally to mean compounds which bear at least one carboxyl group bonded to an aromatic system. Aromatic systems are understood to mean cyclic, through-conjugated systems having (4n+2) π electrons where n is a natural integer and is preferably 1, 2, 3, 4 or 5. The aromatic system may be mono- or polycyclic, for example di- or tricyclic. The aromatic system is preferably formed from carbon atoms. In a further preferred embodiment, as well as carbon atoms, it contains one or more heteroatoms, for example nitrogen, oxygen and/or sulfur. Examples of such aromatic systems are benzene, naphthalene, phenanthrene, furan, pyridine, pyrrole, thiophene and thiazole. The aromatic system may, as well as the ester group, bear one or more, for example one, two, three or more, identical or different further substituents. Suitable further substituents are, for example, alkyl and alkenyl radicals, and also hydroxyl, hydroxyalkyl, alkoxy, poly(alkoxy), amide, cyano, nitrile and/or nitro groups. These substituents may be bonded to any position on the aromatic system. However, the aryl radical bears at most as many substituents as it has valencies.

However, the aryl radical of the aromatic carboxylic ester preferably does not bear any free carboxylic acid or carboxylate groups as further substituents. These could themselves react with the amines of the formula (II) to give unwanted by-products. In a specific embodiment, the aryl radical of the aromatic carboxylic ester (I) bears at least one further, for example two, three, four or more further, carboxylic ester group(s). Thus, the process according to the invention is likewise suitable for conversion of aromatic carboxylic esters having, for example, two, three, four or more ester groups. In the process according to the invention, the ester groups can be converted completely or else only partially to amides. The degree of amidation can be adjusted, for example, through the stoichiometry between carboxylic ester and amine in the reaction mixture.

The process according to the invention is particularly suitable for preparation of alkylarylcarboxamides, for example alkylphenylcarboxamides. In the process according to the invention, aromatic carboxylic esters (I) in which the aryl radical bearing the ester group additionally bears at least one alkyl or alkylene radical are reacted with amines (II). The process is particularly advantageous for preparation of alkylbenzamides whose aryl radical bears at least one alkyl radical having 1 to 20 carbon atoms and especially 1 to 12 carbon atoms, for example 1 to 4 carbon atoms.

The process according to the invention is additionally particularly suitable for preparation of aromatic carboxamides whose aryl radical R³ bears one or more, for example two or three, hydroxyl groups and/or hydroxyalkyl groups. In the reaction of the corresponding esters (I) especially with at most equimolar amounts of amine of the formula (II), selective amidation of the ester group takes place; no aminolysis of the phenolic OH group takes place.

Aromatic esters (I) suitable in accordance with the invention derive, for example, from benzoic acid, phthalic acid, isophthalic acid, the different isomers of naphthalenecarboxylic acid, pyridinecarboxylic acid and naphthalenedicarboxylic acid, and from trimellitic acid, trimesic acid, pyromellitic acid and mellitic acid, the different isomers of methoxybenzoic acid, hydroxybenzoic acid, hydroxymethylbenzoic acid, hydroxymethoxybenzoic acid, hydroxydimethoxybenzoic acid, hydroxyisophthalic acid, hydroxynaphthalenecarboxylic acid, hydroxypyridinecarboxylic acid and hydroxymethylpyridinecarboxylic acid, hydroxyquinolinecarboxylic acid, and from o-toluic acid, m-toluic acid, p-toluic acid, o-ethylbenzoic acid, m-ethylbenzoic acid, p-ethylbenzoic acid, o-propylbenzoic acid, m-propylbenzoic acid, p-propylbenzoic acid and 3,4-dimethylbenzoic acid. Mixtures of different aryl and/or alkylarylcarboxylic esters are equally suitable.

In a preferred embodiment, R⁴ is an aliphatic radical. This has preferably 1 to 24, more preferably 2 to 18 and especially 3 to 6 carbon atoms. The aliphatic radical may be linear or, if it has at least 3 carbon atoms, branched or cyclic. It may additionally be saturated or, if it has at least 3 carbon atoms, unsaturated; it is preferably saturated. The hydrocarbyl radical R⁴ may optionally bear substituents, for example C₅-C₂₀-aryl groups, and/or be interrupted by heteroatoms, for example oxygen and/or nitrogen. Particularly preferred aliphatic radicals are methyl, ethyl, n-propyl, isopropyl, n-butyl, isobutyl and tert-butyl, n-hexyl, cyclohexyl, n-octyl, n-decyl, n-dodecyl, tridecyl, isotridecyl, tetradecyl, hexadecyl, octadecyl and methyl-phenyl.

In a specific embodiment, the esters of the formula (I) derive from alcohols of the formula (IV) whose aliphatic R⁴ radical bears one or more, for example two, three, four, five, six or more, further hydroxyl groups. The hydroxyl groups may be bonded to adjacent carbon atoms or else to further-removed carbon atoms of the hydrocarbyl radical, but at most one OH group per carbon atom. The OH groups of the parent polyols of the esters (I) may be fully or else only partly esterified. They may be esterified with identical or different carboxylic acids. For instance, the process according to the invention is equally suitable for conversion of esters which derive from polyols, for example ethylene glycol, 1,2-propanediol, 1,3-propanediol, neopentyl glycol, glycerol, sorbitol, pentaerythritol, fructose and glucose. The degree of amidation can be controlled, for example, via the stoichiometry between carboxylic ester groups and amino groups in the reaction mixture.

In a further preferred embodiment, R⁴ is an optionally substituted C₅-C₁₂-aryl group or an optionally substituted heteroaromatic group having 5 to 12 ring members. Preferred heteroatoms are oxygen, nitrogen and sulfur. Preferred substituents are, for example, nitro groups. A particularly preferred aromatic R⁴ radical is the nitrophenyl radical.

Examples of suitable alcohols (IV) from which the esters of the formula (I) derive are methanol, ethanol, n-propanol, isopropanol, n-butanol, isobutanol, tert-butanol, pentanol, neopentanol, n-hexanol, isohexanol, cyclohexanol, heptanol, octanol, decanol, dodecanol, tetradecanol, hexadecanol, octadecanol, eicosanol, ethylene glycol, 2-methoxyethanol, propylene glycol, glycerol, sorbitan, sorbitol, diethylene glycol, triethylene glycol, polyethylene glycol, polypropylene glycol, triethanolamine, N,N-dimethylethanolamine, N,N-diethylethanolamine, phenol, naphthol and mixtures thereof. Additionally suitable are fatty alcohol mixtures obtained from natural raw materials, for example coconut fatty alcohol, palm kernel fatty alcohol and tallow fatty alcohol. Particular preference is given to lower aliphatic alcohols such as methanol, ethanol, propanol, n-butanol and glycerol.

Examples of esters of the formula (I) particularly suitable in accordance with the invention are esters of aromatic carboxylic acids and monoalcohols having 1 to 4 carbon atoms.

The process according to the invention is preferentially suitable for preparation of secondary amides. For this purpose, carboxylic esters (I) are reacted with amines (II) in which R¹ is a hydrocarbyl radical having 1 to 100 carbon atoms and R² is hydrogen.

The process according to the invention is additionally more preferably suitable for preparation of tertiary amides. For this purpose, carboxylic esters (I) are reacted with amines (II) in which both R¹ and R² radicals are independently a hydrocarbyl radical having 1 to 100 carbon atoms. The R¹ and R² radicals may be the same or different. In a particularly preferred embodiment, R¹ and R² are the same.

In a first preferred embodiment, R¹ and/or R² are each independently an aliphatic radical. It has preferably 1 to 24, more preferably 2 to 18 and especially 3 to 6 carbon atoms. The aliphatic radical may be linear, branched or cyclic. It may additionally be saturated or unsaturated. The hydrocarbyl radical may bear substituents. Such substituents may be, for example, hydroxyl, C₁-C₅-alkoxy, alkoxyalkyl, cyano, nitrile, nitro and/or C₅-C₂₀-aryl groups, for example phenyl radicals. The C₅-C₂₀-aryl groups may in turn optionally be substituted by halogen atoms, C₁-C₂₀-alkyl, C₂-C₂₀-alkenyl, hydroxyl, C₁-C₅-alkoxy, for example methoxy, ester, amide, cyano, nitrile and/or nitro groups. Particularly preferred aliphatic radicals are methyl, ethyl, hydroxyethyl, n-propyl, isopropyl, hydroxypropyl, n-butyl, isobutyl and tert-butyl, hydroxybutyl, n-hexyl, cyclohexyl, n-octyl, n-decyl, n-dodecyl, tridecyl, isotridecyl, tetradecyl, hexadecyl, octadecyl and methylphenyl. In a particularly preferred embodiment, R¹ and/or R² are each independently hydrogen, a C₁-C₆-alkyl, C₂-C₆-alkenyl or C₃-C₆-cycloalkyl radical, and especially an alkyl radical having 1, 2 or 3 carbon atoms. These radicals may bear up to three substituents.

In a further preferred embodiment, R¹ and R² together with the nitrogen atom to which they are bonded form a ring. This ring has preferably 4 or more, for example 4, 5, 6 or more, ring members. Preferred further ring members are carbon, nitrogen, oxygen and sulfur atoms. The rings may themselves in turn bear substituents, for example alkyl radicals. Suitable ring structures are, for example, morpholinyl, pyrrolidinyl, piperidinyl, imidazolyl and azepanyl radicals.

In a further preferred embodiment, R¹ and/or R² are each independently an optionally substituted C₆-C₁₂-aryl group or an optionally substituted heteroaromatic group having 5 to 12 ring members. Preferred heteroatoms of heteroaromatic groups are oxygen, nitrogen and/or sulfur. Examples of suitable substituents are halogen atoms, halogenated alkyl radicals, and alkyl, alkenyl, hydroxyl, hydroxyalkyl, alkoxy, ester, amide, nitrile and nitro groups.

In a further preferred embodiment, R¹ and/or R² are each independently an alkyl radical interrupted by heteroatoms. Particularly preferred heteroatoms are oxygen and nitrogen.

For instance, R¹ and/or R² are preferably each independently radicals of the formula (V)

—(R⁷O)ₙ—R⁸ (V)

in which

R⁷ is an alkylene group having 2 to 6 carbon atoms, and preferably having 2 to 4 carbon atoms, for example ethylene, propylene, butylene or mixtures thereof,

R⁸ is hydrogen, a hydrocarbyl radical having 1 to 24 carbon atoms or a group of the formula —R⁷NR¹¹R¹²,

n is a number from 2 to 50, preferably from 3 to 25 and especially from 4 to 10, and

R¹¹, R¹² are each independently an aliphatic radical having 1 to 24 carbon atoms and preferably 2 to 18 carbon atoms, an aryl group or heteroaryl group having 5 to 12 ring members, a poly(oxyalkylene) group having 1 to 50 poly(oxyalkylene) units, where the poly(oxyalkylene) units derive from alkylene oxide units having 2 to 6 carbon atoms or R¹¹ and R¹² together with the nitrogen atom to which they are bonded form a ring having 4, 5, 6 or more ring members.

Additionally preferably, R¹ and/or R² are each independently radicals of the formula (VI)

—[R⁹—N(R¹⁰)]ₘ—(R¹⁰) (VI)

in which

R⁹ is an alkylene group having 2 to 6 carbon atoms and preferably having 2 to 4 carbon atoms, for example ethylene, propylene or mixtures thereof,

each R¹⁰ is independently hydrogen, an alkyl or hydroxyalkyl radical having up to 24 carbon atoms, for example 2 to 20 carbon atoms, a polyoxyalkylene radical —(R⁷—O)ₚ—R⁸, or a polyiminoalkylene radical —[R⁹—N(R¹⁰)]_{q}—(R¹⁰), where R⁷, R⁸, R⁹ and R¹⁰ are each as defined above and q and p are each independently 1 to 50, and

m is a number from 1 to 20 and preferably 2 to 10, for example three, four, five or six. The radicals of the formula IV preferably contain 1 to 50 and especially 2 to 20 nitrogen atoms.

In a specific embodiment, the process according to the invention is suitable for preparing carboxamides which bear tertiary amino groups and are thus basic, by reacting at least one aromatic carboxylic ester (I) with at least one polyamine bearing a primary and/or secondary and at least one tertiary amino group under microwave irradiation in a reaction tube whose longitudinal axis is in the direction of propagation of the microwaves of a monomode microwave applicator to give the basic carboxamide. Tertiary amino groups are understood here to mean structural units in which one nitrogen atom does not bear an acidic proton. For example, the nitrogen of the tertiary amino group may bear three hydrocarbyl radicals or else be part of a heterocyclic system. In this embodiment, R¹ preferably has one of the definitions given above, and is more preferably hydrogen, an aliphatic radical having 1 to 24 carbon atoms or an aryl group having 6 to 12 carbon atoms, and especially methyl, and R² is a hydrocarbyl radical which bears tertiary amino groups and is of the formula (VII)

-(A)ₛZ (VII)

in which

A is an alkylene radical having 1 to 12 carbon atoms, a cycloalkylene radical having 5 to 12 ring members, an arylene radical having 6 to 12 ring members or a heteroarylene ring having 5 to 12 ring members,

s is 0 or 1,

Z is a group of the formula —NR¹³R¹⁴ or a nitrogen-containing cyclic hydrocarbyl radical having at least 5 ring members and

R¹³ and R¹⁴ are each independently C₁ to C₂₀ hydrocarbyl radicals, or polyoxyalkylene radicals of the formula —(R⁷—O)—R⁸ (III) where R⁷, R⁸ and p are each as defined above.

A is preferably an alkylene radical having 2 to 24 carbon atoms, a cycloalkylene radical having 5 to 12 ring members, an arylene radical having 6 to 12 ring members or a heteroarylene radical having 5 to 12 ring members. A is more preferably an alkylene radical having 2 to 12 carbon atoms. s is preferably 1. More preferably, A is a linear or branched alkylene radical having 1 to 6 carbon atoms and s is 1.

A is additionally preferably, when Z is a group of the formula —NR¹³R¹⁴, a linear or branched alkylene radical having 2, 3 or 4 carbon atoms, especially an ethylene radical or a linear propylene radical. When Z, in contrast, is a nitrogen-containing cyclic hydrocarbyl radical, particular preference is given to compounds in which A is a linear alkylene radical having 1, 2 or 3 carbon atoms, especially a methylene, ethylene or linear propylene radical.

Cyclical radicals preferred for the structural element A may be mono- or polycyclic and contain, for example, two or three ring systems. Preferred ring systems have 5, 6 or 7 ring members. They preferably contain a total of about 5 to 20 carbon atoms, especially 6 to 10 carbon atoms. Preferred ring systems are aromatic and contain only carbon atoms. In a specific embodiment, the structural elements A are formed from arylene radicals. The structural element A may bear substituents, for example alkyl radicals, nitro, cyano, nitrile, oxyacyl and/or hydroxyalkyl groups. When A is a monocyclic aromatic hydrocarbon, the amino groups or substituents bearing amino groups are preferably in ortho or para positions to one another.

Z is preferably a group of the formula —NR¹³R¹⁴.R¹³ and R¹⁴ therein are preferably each independently aliphatic, aromatic and/or araliphatic hydrocarbyl radicals having 1 to 20 carbon atoms. Particularly preferred as R¹³ and R¹⁴ are alkyl radicals. When R¹³ and/or R¹⁴ are alkyl radicals, they preferably bear 1 to 14 carbon atoms, for example 1 to 6 carbon atoms. These alkyl radicals may be linear, branched and/or cyclic. R¹³ and R¹⁴ are more preferably each alkyl radicals having 1 to 4 carbon atoms, for example methyl, ethyl, n-propyl, isopropyl, n-butyl and isobutyl. In a further embodiment, the R¹³ and/or R¹⁴ radicals are each independently polyoxyalkylene radicals of the formula (III).

Aromatic radicals particularly suitable as R¹³ and/or R¹⁴ include ring systems having at least 5 ring members. They may contain heteroatoms such as S, O and N. Araliphatic radicals particularly suitable as R¹³ and/or R¹⁴ include ring systems which have at least 5 ring members and are bonded to the nitrogen via a C₁-C₆ alkyl radical. They may contain heteroatoms such as S, O and N. The aromatic and also araliphatic radicals may bear further substituents, for example alkyl radicals, nitro, cyano, nitrile, oxyacyl and/or hydroxyalkyl groups.

In a further preferred embodiment, Z is a nitrogen-containing cyclic hydrocarbyl radical whose nitrogen atom is not capable of forming amides. The cyclic system may be mono-, di- or else polycyclic. It preferably contains one or more five- and/or six-membered rings. This cyclic hydrocarbon may contain one or more, for example two or three, nitrogen atoms which do not bear acidic protons; it more preferably comprises one nitrogen atom. Particularly suitable are nitrogen-containing aromatics whose nitrogen is involved in the formation of an aromatic π-electron sextet, for example pyridine. Likewise suitable are nitrogen-containing heteroaliphatics whose nitrogen atoms do not bear protons and whose valences are, for example, all satisfied with alkyl radicals. Z is joined to A or to the nitrogen of the formula (II) here preferably via a nitrogen atom of the heterocycle, as, for example, in the case of 1-(3-aminopropyl)pyrrolidine. The cyclic hydrocarbon represented by Z may bear further substituents, for example C₁-C₂₀-alkyl radicals, halogen atoms, halogenated alkyl radicals, nitro, cyano, nitrile, hydroxyl and/or hydroxyalkyl groups.

According to the stoichiometric ratio between carboxylic ester (I) and polyamine (VI), one or more amino groups which each bear at least one hydrogen atom are converted to the carboxamide. In the reaction of polycarboxylic esters with amines of the formula (II), as also with polyamines of the formula (VI), primary amino groups can also be converted to imides.

For the inventive preparation of primary amides, instead of ammonia, preference is given to using nitrogen compounds which eliminate ammonia gas when heated. Examples of such nitrogen compounds are urea and formamide.

Examples of suitable amines are ammonia, methylamine, ethylamine, propylamine, butylamine, hexylamine, cyclohexylamine, octylamine, decylamine, dodecylamine, tetradecylamine, hexadecylamine, octadecylamine, dimethylamine, diethylamine, ethylmethylamine, di-n-propylamine, diisopropylamine, dicyclohexylamine, didecylamine, didodecylamine, ditetradecylamine, dihexadecylamine, dioctadecylamine, benzylamine, phenylethylamine, ethylenediamine, diethylenetriamine, triethylenetetramine, tetraethylenepentamine and mixtures thereof. Examples of suitable amines bearing tertiary amine groups are N,N-dimethylethylenediamine, N,N-dimethyl-1,3-propanediamine, N,N-diethyl-1,3-propanediamine, N,N-dimethyl-2-methyl-1,3-propanediamine, 1-(3-aminopropyl)pyrrolidine, 1-(3-aminopropyl)-4-methylpiperazine, 3-(4-morpholino)-1-propylamine, 2-aminothiazole, the different isomers of N,N-dimethylaminoaniline, of aminopyridine, of aminomethylpyridine, of aminomethylpiperidine and of aminoquinoline, and also 2-aminopyrimidine, 3-aminopyrazole, aminopyrazine and 3-amino-1,2,4-triazole. Mixtures of different amines are also suitable. Among these, particular preference is given to dimethylamine, diethylamine, di-n-propylamine, diisopropylamine, ethylmethylamine and N,N-dimethylaminopropylamine.

Particular preference is given to the process according to the invention for preparing amides of aliphatic C₁-C₄-alkylamines by reacting esters of aromatic carboxylic acids and aliphatic C₁-C₆-alcohols with primary or secondary aliphatic C₁-C₄-alkylamines. Particular preference is additionally given to the process according to the invention for preparing basic amides by reacting esters of aromatic carboxylic acids and lower aliphatic C₁-C₄-alcohols with polyamines bearing at least one primary and/or secondary and at least one tertiary amino group.

In the case that the carboxylic ester (I) contains two or more ester groups and the amine (II) two or more amino groups, or both reactants each bear one ester and one amino group, it is also possible by the process according to the invention to prepare polymers. In this case, the rising viscosity of the reaction mixture during the microwave irradiation should be noted in the design of the apparatus. The process is especially suitable for preparing N,N-dimethylbenzamide, N,N-diethylbenzamide, N,N-(2-hydroxyethyl)benzamide, N-(N′,N″-dimethylamino)propylbenzamide, N,N-dimethylnicotinamide, N,N-dimethyltoluamide and N-(N″,N″-dimethylamino)propyltoluamide, and mixtures thereof.

In the process according to the invention, it is possible to react aromatic carboxylic ester and amine with one another in any desired ratios. Preference is given to effecting the reaction between ester and amine with molar ratios of 10:1 to 1:100, preferably of 2:1 to 1:10, especially of 1.2:1 to 1:3, based in each case on the molar equivalents of ester and amine groups. In a specific embodiment, ester and amine are used in equimolar amounts.

In the case that the aromatic hydrocarbyl radical R³ bears one or more hydroxyl groups, the reaction between carboxylic ester (I) and amine (II) is preferably effected with molar ratios of 100:1 to 1:1, preferably of 10:1 to 1.001:1 and especially of 5:1 to 1.01:1, for example of 2:1 to 1.1:1, based in each case on the molar equivalents of ester groups and amino groups in the reaction mixture.

In many cases, it has been found to be advantageous to work with an excess of amine, i.e. molar ratios of amine to ester of at least 1.01:1.00 and especially between 50:1 and 1.02:1, for example between 10:1 and 1.1:1. The ester groups are converted virtually quantitatively to the amide. This process is particularly advantageous when the amine used is volatile. “Volatile” means here that the amine has a boiling point at standard pressure of preferably below 200° C. and more preferably below 160° C., for example below 100° C., and can thus be removed from the amide by distillation.

In a preferred embodiment, the reaction is accelerated or completed by working in the presence of catalysts. Preference is given to working in the presence of a basic catalyst or mixtures of two or more of these catalysts. The basic catalysts used in the context of the present invention are quite generally those basic compounds which are suitable for accelerating the amidation of carboxylic esters with amines to give carboxamides. Examples of suitable catalysts are inorganic and organic bases, for example metal hydroxides, oxides, carbonates or alkoxides. In a preferred embodiment, the basic catalyst is selected from the group of the hydroxides, oxides, carbonates and alkoxides of alkali metals and alkaline earth metals. Very particular preference is given to lithium hydroxide, sodium hydroxide, potassium hydroxide, sodium methoxide, potassium methoxide, sodium carbonate and potassium carbonate. Cyanide ions are also suitable as a catalyst. These substances can be used in solid form or as a solution, for example as an alcoholic solution. The amount of the catalysts used depends on the activity and stability of the catalyst under the selected reaction conditions and should be matched to the particular reaction. The amount of the catalyst to be used can vary within wide limits. It has often been found to be useful to work with 0.1 to 2.0 mol of base, for example with 0.2 to 1.0 mol of base, per mole of amine used. Particular preference is given to using catalytic amounts of the abovementioned reaction-accelerating compounds, preferably in the range between 0.001 and 10% by weight, more preferably in the range from 0.01 to 5% by weight, for example between 0.02 and 2% by weight, based on the amount of carboxylic ester and amine used.

The inventive preparation of the amides proceeds by mixing carboxylic ester and amine and optionally catalyst and then irradiating the reaction mixture with microwaves in a reaction tube whose longitudinal axis is in the direction of propagation of the microwaves in a monomode microwave applicator.

The reaction mixture is preferably irradiated with microwaves in a substantially microwave-transparent reaction tube within a hollow conductor connected to a microwave generator. The reaction tube is preferably aligned axially with the central axis of symmetry of the hollow conductor.

The hollow conductor which functions as the microwave applicator is preferably configured as a cavity resonator. Additionally preferably, the microwaves unabsorbed in the hollow conductor are reflected at the end thereof. The length of the cavity resonator is preferably such that a standing wave forms therein. Configuration of the microwave applicator as a resonator of the reflection type achieves a local increase in the electrical field strength at the same power supplied by the generator and increased energy exploitation.

The cavity resonator is preferably operated in E₀₁ₙ mode where n is an integer and specifies the number of field maxima of the microwave along the central axis of symmetry of the resonator. In this operation, the electrical field is directed in the direction of the central axis of symmetry of the cavity resonator. It has a maximum in the region of the central axis of symmetry and decreases to the value 0 toward the outer surface. This field configuration is rotationally symmetric about the central axis of symmetry. Use of a cavity resonator with a length where n is an integer enables the formation of a standing wave. According to the desired flow rate of the reaction mixture through the reaction tube, the temperature required and the residence time required in the resonator, the length of the resonator is selected relative to the wavelength of the microwave radiation used. n is preferably an integer from 1 to 200, more preferably from 2 to 100, particularly from 3 to 50, especially from 4 to 20, for example three, four, five, six, seven, eight, nine or ten.

The E₀₁ₙ mode of the cavity resonator is also referred to in English as the TM₀₁ₙ mode; see, for example, K. Lange, K. H. Löcherer, “Taschenbuch der Hochfrequenztechnik” [Handbook of High-Frequency Technology], volume 2, pages K21 ff.

The microwave energy can be injected into the hollow conductor which functions as the microwave applicator through holes or slots of suitable dimensions. In an embodiment particularly preferred in accordance with the invention, the reaction mixture is irradiated with microwaves in a reaction tube present in a hollow conductor with a coaxial transition of the microwaves. Microwave devices particularly preferred for this process are formed from a cavity resonator, a coupling device for injecting a microwave field into the cavity resonator and with one orifice each on two opposite end walls for passage of the reaction tube through the resonator. The microwaves are preferably injected into the cavity resonator by means of a coupling pin which projects into the cavity resonator. The coupling pin is preferably configured as a preferably metallic inner conductor tube which functions as a coupling antenna. In a particularly preferred embodiment, this coupling pin projects through one of the end orifices into the cavity resonator. The reaction tube more preferably adjoins the inner conductor tube of the coaxial transition, and is especially conducted through the cavity thereof into the cavity resonator. The reaction tube is preferably aligned axially with a central axis of symmetry of the cavity resonator. For this purpose, the cavity resonator preferably has one central orifice each on two opposite end walls for passage of the reaction tube.

The microwaves can be fed into the coupling pin or into the inner conductor tube which functions as a coupling antenna, for example, by means of a coaxial connecting line. In a preferred embodiment, the microwave field is supplied to the resonator via a hollow conductor, in which case the end of the coupling pin projecting out of the cavity resonator is conducted into the hollow conductor through an orifice in the wall of the hollow conductor, and takes microwave energy from the hollow conductor and injects it into the resonator.

In a specific embodiment, the reaction mixture is irradiated with microwaves in a microwave-transparent reaction tube which is axially symmetric within an E₀₁ₙ round hollow conductor with a coaxial transition of the microwaves. In this case, the reaction tube is conducted through the cavity of an inner conductor tube which functions as a coupling antenna into the cavity resonator. In a further preferred embodiment, the reaction mixture is irradiated with microwaves in a microwave-transparent reaction tube which is conducted through an E₀₁ₙ cavity resonator with axial introduction of the microwaves, the length of the cavity resonator being such as to form n=2 or more field maxima of the microwave. In a further preferred embodiment, the reaction mixture is irradiated with microwaves in a microwave-transparent reaction tube which is conducted through an E₀₁ₙ cavity resonator with axial introduction of the microwaves, the length of the cavity resonator being such as to form a standing wave where n=2 or more field maxima of the microwave. In a further preferred embodiment, the reaction mixture is irradiated with microwaves in a microwave-transparent reaction tube which is axially symmetric within a circular cylindrical E₀₁ₙ cavity resonator with a coaxial transition of the microwaves, the length of the cavity resonator being such as to form n=2 or more field maxima of the microwave. In a further preferred embodiment, the reaction mixture is irradiated with microwaves in a microwave-transparent reaction tube which is axially symmetric within a circular cylindrical E₀₁ₙ cavity resonator with a coaxial transition of the microwaves, the length of the cavity resonator being such as to form a standing wave where n=2 or more field maxima of the microwave.

Microwave generators, for example the magnetron, the klystron and the gyrotron, are known to those skilled in the art.

The reaction tubes used to perform the process according to the invention are preferably manufactured from substantially microwave-transparent, high-melting material. Particular preference is given to using nonmetallic reaction tubes. “Substantially microwave-transparent” is understood here to mean materials which absorb a minimum amount of microwave energy and convert it to heat. A measure employed for the ability of a substance to absorb microwave energy and convert it to heat is often the dielectric loss factor tan δ=ε″/ε′. The dielectric loss factor tan δ is defined as the ratio of dielectric loss ε″ to dielectric constant ε′. Examples of tan δ values of different materials are reproduced, for example, in D. Bogdal, Microwave-assisted Organic Synthesis, Elsevier 2005. For reaction tubes suitable in accordance with the invention, materials with tan δ values measured at 2.45 GHz and 25° C. of less than 0.01, particularly less than 0.005 and especially less than 0.001 are preferred. Preferred microwave-transparent and thermally stable materials include primarily mineral-based materials, for example quartz, aluminum oxide, sapphire, zirconium oxide, silicon nitride and the like. Other suitable tube materials are thermally stable plastics, such as especially fluoropolymers, for example Teflon, and industrial plastics such as polypropylene, or polyaryl ether ketones, for example glass fiber-reinforced polyetheretherketone (PEEK). In order to withstand the temperature conditions during the reaction, minerals, such as quartz or aluminum oxide, coated with these plastics have been found to be especially suitable as reactor materials.

Reaction tubes particularly suitable for the process according to the invention have an internal diameter of one millimeter to approx. 50 cm, particularly between 2 mm and 35 cm, especially between 5 mm and 15 cm, for example between 10 mm and 7 cm. Reaction tubes are understood here to mean vessels whose ratio of length to diameter is greater than 5, preferably between 10 and 100 000, more preferably between 20 and 10 000, for example between 30 and 1000. The length of the reaction tube is understood here to mean the length of the reaction tube over which the microwave irradiation proceeds. Baffles and/or other mixing elements can be incorporated into the reaction tube.

E₀₁ cavity resonators particularly suitable for the process according to the invention preferably have a diameter which corresponds to at least half the wavelength of the microwave radiation used. The diameter of the cavity resonator is preferably 1.0 to 10 times, more preferably 1.1 to 5 times and especially 2.1 to 2.6 times half the wavelength of the microwave radiation used. The E₀₁ cavity resonator preferably has a round cross section, which is also referred to as an E₀₁ round hollow conductor. It more preferably has a cylindrical shape and especially a circular cylindrical shape.

The reaction tube is typically provided at the inlet with a metering pump and a manometer, and at the outlet with a pressure-retaining device and a heat exchanger. This makes possible reactions within a very wide pressure and temperature range.

The preparation of the reaction mixture from ester, amine and optionally catalyst and/or solvent can be performed continuously, batchwise or else in semibatchwise processes. Thus, the reaction mixture can be prepared in an upstream (semi)batchwise process, for example in a stirred vessel. In a preferred embodiment, the amine and carboxylic ester reactants, each independently optionally diluted with solvent, are only mixed shortly before entry into the reaction tube. For instance, it has been found to be particularly useful, when using reactants which do not have unlimited mutual miscibility, to undertake the mixing of amine and ester in a mixing zone, from which the reaction mixture is conveyed into the reaction tube. Additionally preferably, the reactants are supplied to the process according to the invention in liquid form. For this purpose, it is possible to use relatively high-melting and/or relatively high-viscosity reactants, for example in the molten state and/or admixed with solvent, for example in the form of a solution, dispersion or emulsion. A catalyst can, if used, be added to one of the reactants or else to the reactant mixture before entry into the reaction tube. Preference is given to using catalysts in liquid form, for example as a solution in one of the reactants or in a solvent which is inert under the reaction conditions. It is also possible to convert heterogeneous systems by the process according to the invention, in which case merely appropriate industrial apparatus for conveying the reaction mixture is required.

The reaction mixture can be fed into the reaction tube either at the end conducted through the inner conductor tube or at the opposite end. The reaction mixture can consequently be conducted in a parallel or antiparallel manner to the direction of propagation of the microwaves through the microwave applicator.

By variation of tube cross section, length of the irradiation zone (this is understood to mean the length of the reaction tube in which the reaction mixture is exposed to microwave radiation), flow rate, geometry of the cavity resonator and the microwave power injected, the reaction conditions are preferably established such that the maximum reaction temperature is attained as rapidly as possible and the residence time at maximum temperature remains sufficiently short that as low as possible a level of side reactions or further reactions occurs. To complete the reaction, the reaction mixture can pass through the reaction tube more than once, optionally after intermediate cooling. In many cases, it has been found to be useful when the reaction product is cooled immediately after leaving the reaction tube, for example by jacket cooling or decompression. In the case of slower reactions, it has often been found to be useful to keep the reaction product at reaction temperature for a certain time after it leaves the reaction tube.

The temperature rise caused by the microwave irradiation is preferably limited to a maximum of 500° C., for example, by regulating the microwave intensity or the flow rate and/or by cooling the reaction tube, for example by means of a nitrogen stream. It has been found to be particularly useful to perform the reaction at temperatures between 120 and a maximum of 400° C., particularly between 135 and a maximum of 350° C. and especially between 155 and a maximum of 300° C., for example at temperatures between 180 and 270° C.

The duration of the microwave irradiation depends on various factors, for example the geometry of the reaction tube, the microwave energy injected, the specific reaction and the desired degree of conversion. Typically, the microwave irradiation is undertaken over a period of less than 30 minutes, preferably between 0.01 second and 15 minutes, more preferably between 0.1 second and 10 minutes and especially between 1 second and 5 minutes, for example between 5 seconds and 2 minutes. The intensity (power) of the microwave radiation is adjusted such that the reaction mixture has the desired maximum temperature when it leaves the cavity resonator. In a preferred embodiment, the reaction product, directly after the microwave irradiation has ended, is cooled as rapidly as possible to temperatures below 120° C., preferably below 100° C. and especially below 60° C. In a further preferred embodiment, the catalyst, if present, is neutralized directly after leaving the reaction tube.

The reaction is preferably performed at pressures between atmospheric pressure and 500 bar, more preferably between 1.5 bar and 150 bar, particularly between 3 bar and 100 bar and especially between 5 bar and 100 bar, for example between 10 bar and 50 bar. It has been found to be particularly useful to work under elevated pressure, which involves working above the boiling point (at standard pressure) of the reactants, products, any solvent present, and/or above the alcohol formed during the reaction. The pressure is more preferably adjusted to a sufficiently high level that the reaction mixture remains in the liquid state during the microwave irradiation and does not boil.

To avoid side reactions and to prepare products of maximum purity, it has been found to be useful to handle reactants and products in the presence of an inert protective gas, for example nitrogen, argon or helium.

It has been found to be useful to work in the presence of solvents in order, for example, to lower the viscosity of the reaction medium and/or to fluidize the reaction mixture if it is heterogeneous. For this purpose, it is possible in principle to use all solvents which are inert under the reaction conditions employed and do not react with the reactants or the products formed. An important factor in the selection of suitable solvents is the polarity thereof, which firstly determines the dissolution properties and secondly the degree of interaction with microwave radiation. A particularly important factor in the selection of suitable solvents is the dielectric loss ε″ thereof. The dielectric loss ε″ describes the proportion of microwave radiation which is converted to heat in the interaction of a substance with microwave radiation. The latter value has been found to be a particularly important criterion for the suitability of a solvent for the performance of the process according to the invention.

It has been found to be particularly useful to work in solvents which exhibit minimum microwave absorption and hence make only a small contribution to the heating of the reaction system. Solvents preferred for the process according to the invention have a dielectric loss ε″ measured at room temperature and 2450 MHz of less than 10 and preferably less than 1, for example less than 0.5. An overview of the dielectric loss of different solvents can be found, for example, in “Microwave Synthesis” by B. L. Hayes, CEM Publishing 2002. Suitable solvents for the process according to the invention are especially those with ε″ values less than 10, such as N-methylpyrrolidone, N,N-dimethylformamide or acetone, and especially solvents with ε″ values less than 1. Examples of particularly preferred solvents with ε″ values less than 1 are aromatic and/or aliphatic hydrocarbons, for example toluene, xylene, ethylbenzene, tetralin, hexane, cyclohexane, decane, pentadecane, decalin, and also commercial hydrocarbon mixtures, such as benzine fractions, kerosene, Solvent Naphtha, Shellsol® AB, Solvesso® 150, Solvesso® 200, Exxsol®, Isopar® and Shellsol® products. Solvent mixtures which have ε41 values preferably below 10 and especially below 1 are equally preferred for the performance of the process according to the invention.

In a further preferred embodiment, the process according to the invention is performed in solvents with higher ε″ values of, for example, 5 or higher, such as especially with ε″ values of 10 or higher. This embodiment has been found to be useful especially in the conversion of reaction mixtures which themselves, i.e. without the presence of solvents and/or diluents, exhibit only very low microwave absorption. For instance, this embodiment has been found to be useful especially in the case of reaction mixtures which have a dielectric loss ε″ of less than 10 and preferably less than 1. However, the accelerated heating of the reaction mixture often observed as a result of the solvent addition entails measures to comply with the maximum temperature.

When working in the presence of solvents, the proportion thereof in the reaction mixture is preferably between 2 and 95% by weight, especially between 5 and 90% by weight and particularly between 10 and 75% by weight, for example between 30 and 60% by weight. Particular preference is given to performing the reaction without solvents.

In a further preferred embodiment, substances which have strong microwave absorption and are insoluble in the reaction mixture are added thereto. These lead to significant local heating of the reaction mixture and, as a result, to further-accelerated reactions. One example of a suitable heat collector of this kind is graphite.

Microwaves refer to electromagnetic rays with a wavelength between about 1 cm and 1 m, and frequencies between about 300 MHz and 30 GHz. This frequency range is suitable in principle for the process according to the invention. For the process according to the invention, preference is given to using microwave radiation with the frequencies approved for industrial, scientific, medical, domestic or similar applications, for example with frequencies of 915 MHz, 2.45 GHz, 5.8 GHz or 24.12 GHz.

The microwave power to be injected into the cavity resonator for the performance of the process according to the invention is especially dependent on the target reaction temperature, but also on the geometry of the reaction tube and hence of the reaction volume, and on the duration of the irradiation required. It is typically between 200 W and several hundred kW and especially between 500 W and 100 kW, for example between 1 kW and 70 kW. It can be generated by means of one or more microwave generators.

In a preferred embodiment, the reaction is performed in a pressure-resistant, chemically inert tube, in which case it is possible that the reactants and products and, if present, solvent can lead to a pressure buildup. After the reaction has ended, the elevated pressure can be used, by decompression, for volatilization and removal of volatile components and any solvent and/or to cool the reaction product. The alcohol formed as a by-product can, after cooling and/or decompression, be removed by customary processes, for example phase separation, distillation, stripping, flashing and/or absorption. The alcohol can often also remain in the product.

To achieve particularly high conversions, it has in many cases been found to be useful to expose the reaction product obtained, optionally after removal of product and/or by-product, again to microwave irradiation, in which case the ratio of the reactants used may have to be supplemented to replace consumed or deficient reactants.

Typically, amides prepared via the inventive route are obtained in a purity sufficient for further use. For specific requirements, they can, however, be purified further by customary purifying processes, for example distillation, recrystallization, filtration or chromatographic processes.

The advantages of the process according to the invention lie in very homogeneous irradiation of the reaction mixture in the center of a symmetric microwave field within a reaction tube, the longitudinal axis of which is in the direction of propagation of the microwaves of a monomode microwave applicator and especially within an E₀₁ cavity resonator, for example with a coaxial transition. The inventive reactor design allows the performance of reactions also at very high pressures and/or temperatures. By increasing the temperature and/or pressure, a significant rise in the degree of conversion and yield is observed even compared to known microwave reactors, without this resulting in undesired side reactions and/or discoloration. Surprisingly, a very high efficiency is achieved in the exploitation of the microwave energy injected into the cavity resonator, which is typically above 50%, often above 80%, in some cases above 90% and in special cases above 95%, for example above 98%, of the microwave power injected, and thus gives economic and environmental advantages over conventional preparation processes, and also over prior art microwave processes.

The process according to the invention additionally allows a controlled, reliable and reproducible reaction regime. Since the reaction mixture in the reaction tube is moved parallel to the direction of propagation of the microwaves, known overheating phenomena resulting from uncontrollable field distributions, which lead to local overheating as a result of changing intensities of the microwave field, for example in wave crests and node points, are balanced out by the flowing motion of the reaction mixture. The advantages mentioned also allow working with high microwave powers of, for example, more than 10 kW or more than 100 kW, and hence, in combination with only a short residence time in the cavity resonator, accomplishment of large production volumes of 100 or more tonnes per year in one plant.

It was surprising that, in spite of the only very short residence time of the reaction mixture in the microwave field in the flow tube with continuous flow, very substantial amidation with conversions generally of more than 80%, often even more than 90%, for example more than 95%, based on the component used in deficiency, takes place without formation of significant amounts of by-products. It was additionally surprising that the high conversions mentioned can be achieved under these reaction conditions without removal of the alcohol formed in the aminolysis. In case of a corresponding conversion of these reaction mixtures in a flow tube of the same dimensions with thermal jacket heating, extremely high wall temperatures are required to achieve suitable reaction temperatures, which lead to the formation of colored species, but bring about only slight amide formation within the same time interval.

The process according to the invention thus allows very rapid, energy-saving and inexpensive preparation of carboxamides in high yields and with high purity in industrial scale amounts. In this process—aside from the alcohol—no significant amounts of by-products are obtained. Such rapid and selective conversions are unachievable by conventional methods and were not to be expected solely through heating to high temperatures.

### EXAMPLES

The conversions of the reaction mixtures under microwave irradiation were effected in a ceramic tube (60×1 cm) which was present in axial symmetry in a cylindrical cavity resonator (60×10 cm). On one of the end sides of the cavity resonator, the ceramic tube passed through the cavity of an inner conductor tube which functions as a coupling antenna. The microwave field with a frequency of 2.45 GHz, generated by a magnetron, was injected into the cavity resonator by means of the coupling antenna (E₀₁ cavity applicator; monomode), in which a standing wave formed.

The microwave power was in each case adjusted over the experiment time in such a way that the desired temperature of the reaction mixture at the end of the irradiation zone was kept constant. The microwave powers mentioned in the experiment descriptions therefore represent the mean value of the microwave power injected over time. The measurement of the temperature of the reaction mixture was undertaken directly after it had left the reaction zone (distance about 15 cm in an insulated stainless steel capillary, φ1 cm) by means of a Pt100 temperature sensor. Microwave energy not absorbed directly by the reaction mixture was reflected at the opposite end of the cavity resonator from the coupling antenna; the microwave energy which was also not absorbed by the reaction mixture on the return path and reflected back in the direction of the magnetron was passed with the aid of a prism system (circulator) into a water-containing vessel. difference between energy injected and heating of this water load was used to calculate the microwave energy introduced into the reaction mixture.

By means of a high-pressure pump and of a suitable pressure-release valve, the reaction mixture in the reaction tube was placed under such a working pressure which was sufficient always to keep all reactants and products or condensation products in the liquid state. The reaction mixtures comprising ester and amine were pumped with a constant flow rate through the reaction tube, and the residence time in the irradiation zone was adjusted by modifying the flow rate.

The products were analyzed by means of ¹H NMR spectroscopy at 500 MHz in CDCl₃.

### Example 1

### Preparation of N,N-diethyl-m-toluamide

A 10 l Büchi stirred autoclave with gas inlet tube, stirrer, internal thermometer and pressure equalizer was initially charged with 2.4 kg (12.5 mol) of butyl m-toluate, 3.66 kg of diethylamine (50 mol) and 100 g of sodium ethoxide as a catalyst were added gradually, and the mixture was homogenized while stirring.

The reaction mixture thus obtained was pumped through the reaction tube continuously at 5 l/h at a working pressure of 35 bar and exposed to a microwave power of 2.8 kW, 90% of which was absorbed by the reaction mixture. The residence time of the reaction mixture in the irradiation zone was approx. 34 seconds. At the end of the reaction tube, the reaction mixture had a temperature of 305° C. Immediately after leaving the reactor, the reaction mixture was cooled to room temperature.

A conversion of 76% of theory was attained. The reaction product had a pale yellowish color. After distillative removal of the alcohol formed and of the excess or unconverted reactants, 1.8 kg of N,N-diethyl-m-toluamide with a purity of 98% were obtained by means of vacuum distillation.

### Example 2

### Preparation of N-(3-N,N-dimethylamino)propyl-m-toluamide

A 10 l Büchi stirred autoclave with gas inlet tube, stirrer, internal thermometer and pressure equalizer was initially charged with 2.3 kg of butyl m-toluate (12 mol). 3.7 kg of dimethylaminopropylamine (36 mol) and 100 g of sodium ethoxide as a catalyst are added gradually thereto, and the mixture is homogenized while stirring.

The reaction mixture thus obtained was pumped through the reaction tube continuously at 3.8 l/h at a working pressure of 25 bar and exposed to a microwave power of 2.35 kW, 94% of which was absorbed by the reaction mixture. The residence time of the reaction mixture in the irradiation zone was approx. 45 seconds. At the end of the reaction tube, the reaction mixture had a temperature of 285° C. Immediately after leaving the reactor, the reaction mixture was cooled to room temperature with a jacketed coil heat exchanger.

A conversion of 82% of theory was attained. The reaction product was yellowish; the iron content was <5 ppm. After distillative removal of the butanol formed and of the excess or unconverted reactants, 2.1 kg of N-(3-N,N-dimethylamino)propyl-m-toluamide with a purity of 98% were obtained by means of vacuum distillation.

### Example 3

### Preparation of N,N-diethylnicotinamide

A 10 l Büchi stirred autoclave with gas inlet tube, stirrer, internal thermometer and pressure equalizer was initially charged with 1.72 kg of methyl nicotinate (12.5 mol). 3.68 kg of diethylamine (50 mol) and 100 g of potassium tert-butoxide as a catalyst were slowly added thereto, and the mixture was homogenized while stirring.

The reaction mixture thus obtained was pumped through the reaction tube continuously at 4 l/h at a working pressure of 30 bar and exposed to a microwave power of 2.8 kW, 91% of which was absorbed by the reaction mixture. The residence time of the reaction mixture in the irradiation zone was approx. 42 seconds. At the end of the reaction tube, the reaction mixture had a temperature of 285° C. Immediately after leaving the reactor, the reaction mixture was cooled to room temperature with a jacketed coil heat exchanger.

A conversion of 85% of theory was attained. The reaction product was pale yellowish in color; the iron content was <5 ppm. After distillative removal of the methanol formed and of the excess or unconverted reactants, 1.85 kg of N,N-diethylnicotinamide with a purity of 98% were obtained by means of vacuum distillation.

### Example 4

### Preparation of N-stearylphthalimide

A 10 l Büchi stirred autoclave with gas inlet tube, stirrer, internal thermometer and pressure equalizer was initially charged with 3 liters of Solvesso® 150 and 2.3 kg of dimethyl phthalate (12 mol). 2.56 kg of molten stearylamine (10 mol) and 100 g of sodium methoxide as a catalyst were added gradually to this mixture at 50-60° C., and the mixture was homogenized while stirring.

The reaction mixture thus obtained was pumped through the reaction tube continuously at 4.5 l/h at a working pressure of 35 bar and exposed to a microwave power of 3.4 kW, 88% of which was absorbed by the reaction mixture. The residence time of the reaction mixture in the irradiation zone was approx. 37 seconds. At the end of the reaction tube, the reaction mixture had a temperature of 265° C. Immediately after leaving the reactor, the reaction mixture was cooled to room temperature with a jacketed coil heat exchanger.

A conversion of 95% of theory was attained. The reaction product was pale yellowish in color; the iron content was <5 ppm. After distillative removal of solvent and methanol formed, and washing of the crude product with dilute acetic acid and water to neutralize the catalyst, and removal of unconverted reactants, 3.5 kg of N-stearylphthalimide with a purity of 97% were obtained.

1. A continuous process for preparing an amide of an aromatic carboxylic acid, in which at least one carboxylic ester of the formula (I)
R³—COOR⁴ (I)
in which
R³ is an optionally substituted aromatic hydrocarbyl radical having 5 to 100 carbon atoms and
R⁴ is an optionally substituted hydrocarbyl radical having 1 to 30 carbon atoms is reacted with at least one amine of the formula (II)
HNR¹R² (II)
in which R¹ and R² are each independently hydrogen or an optionally substituted hydrocarbyl radical having 1 to 100 carbon atoms, and where R¹ and R² together with the nitrogen atom to which they are bonded may form a ring,
under microwave irradiation of the reaction mixture in a substantially microwave-transparent reaction tube within a hollow conductor which is connected via wave-guides to a microwave generator and whose longitudinal axis is in the direction of propagation of the microwaves of a monomode microwave applicator to give the carboxamide.

2. A process as claimed in claim 1, in which the microwave applicator is configured as a cavity resonator.

3. A process as claimed in claim 1, in which the microwave applicator is configured as a cavity resonator of the reflection type.

4. A process as claimed in claim 1, in which the reaction tube is aligned axially with a central axis of symmetry of the hollow conductor.

5. A process as claimed in claim 2, in which the reaction mixture is irradiated in a cavity resonator with a coaxial transition of the microwaves.

6. A process as claimed in claim 2, in which the cavity resonator is operated in E₀₁ₙ mode where n is an integer from 1 to 200.

7. A process as claimed in claim 2, in which a standing wave forms in the cavity resonator.

8. A process as claimed in claim 1, in which the reaction mixture is heated by the microwave irradiation to temperatures between 120 and 500° C.

9. A process as claimed in claim 1, in which the microwave irradiation is effected at pressures above atmospheric pressure.

10. A process as claimed in claim 1, in which R³ is an optionally substituted cyclic through-conjugated system having (4n+2) π electrons where n is 1, 2, 3, 4 or 5.

11. A process as claimed in claim 1, in which R³ bears at least one further ester group —COOR⁴ in which R⁴ is an optionally substituted hydrocarbyl radical having 1 to 30 carbon atoms.

12. A process as claimed in claim 1, in which R⁴ comprises 2 to 24 carbon atoms.

13. A process as claimed in claim 1, in which R⁴ has at least one further hydroxyl group.

14. A process as claimed in claim 1, in which the compound of the formula (I) is an ester of an aromatic carboxylic acid with a monoalcohol having 1 to 4 carbon atoms.

15. A The process as claimed in claim 1, in which R¹ and/or R² are each independently optionally substituted aliphatic radicals having 2 to 24 carbon atoms.

16. A process as claimed in claim 1, in which R¹ and R² together with the nitrogen atom to which they are bonded form a ring having 4 or more ring members.

17. A process as claimed in claim 1, in which R¹ and/or R² are each independently an optionally substituted C₆-C₁₂-aryl group or an optionally substituted heteroaromatic group having 5 to 12 ring members.

18. A process as claimed in claim 1, in which R¹ and/or R² are each independently radicals of the formula (V)
—(R⁷O)ₙ—R⁸ (V)
in which
R⁷ is an alkylene group having 2 to 6 carbon atoms or mixtures thereof,
R⁸ is hydrogen, a hydrocarbyl radical having 1 to 24 carbon atoms or a group of the formula —R⁷—NR¹¹R¹²,
n is a number from 2 to 50, and
R¹¹, R¹² are each independently an aliphatic radical having 1 to 24 carbon atoms, an aryl or heteroaryl group having 5 to 12 ring members, a poly-(oxyalkylene) group having 1 to 50 poly(oxyalkylene) units, where the poly(oxyalkylene) units derive from alkylene oxide units having 2 to 6 carbon atoms, or R¹¹ and R¹² together with the nitrogen atom to which they are bonded form a ring having 4, 5, 6 or more ring members.

19. A process as claimed in claim 1, in which R¹ and/or R² are each independently radicals of the formula (VI)
—[R⁹—N(R¹⁰)]ₘ—(R¹⁰) (VI)
in which
R⁹ is an alkylene group having 2 to 6 carbon atoms or mixtures thereof,
each R¹⁰ is independently hydrogen, an alkyl or hydroxyalkyl radical having up to 24 carbon atoms, a polyoxyalkylene radical —(R⁷—O)—R⁸ or a polyiminoalkylene radical —[R⁹—N(R¹⁰)]_{q}—(R¹⁰),
R⁷ is an alkylene group having 2 to 6 carbon atoms or mixtures thereof,
R⁸ is hydrogen, a hydrocarbyl radical having 1 to 24 carbon atoms or a group of the formula —R⁷—NR¹¹R¹²,
R¹¹, R¹² are each independently an aliphatic radical having 1 to 24 carbon atoms, an aryl or heteroaryl group having 5 to 12 ring members, a poly(oxyalkylene) group having 1 to 50 poly(oxyalkylene) units, where the poly(oxyalkylene) units derive from alkylene oxide units having 2 to 6 carbon atoms, or R¹¹ and R¹² together with the nitrogen atom to which they are bonded form a ring having 4, 5, 6 or more ring members, and q and p are each independently from 1 to 50 and
m is a number from 1 to 20.

20. A process as claimed in claim 1, in which the amine of the formula (II) is a primary amine.

21. A process as claimed in claim 1, in which the amine of the formula (II) is a secondary amine.
